(19) United States
(12) Patent Application Publication
(10) Pub. No.: US 2026/0240522 A1
(43) Pub. Date: Aug. 20, 2026

(54) ULTRASOUND PROBE AND SYSTEM FOR ANALYZING SUPERFICIAL BODY FLUIDS

(71) Applicant: NEOS NEW BORN SOLUTIONS S.L., Madrid (ES)

(72) Inventors: Javier JIMÉNEZ GONZÁLEZ, Madrid (ES); Ramon SERRANO DELOUNDER, Madrid (ES); Hassane BAGHDAD KICHOU, Madrid (ES); Rita QUESADA DIEZ, Madrid (ES); Guillermo NAVARRO PATRÓN, Madrid (ES); Manuel NAVARRETE HERNÁNDEZ, Madrid (ES); Beatrice JOBST, Madrid (ES); Francesc CARANDELL VERDAGUER, Madrid (ES); Roberto Fabião SANTOS ABREU, Madrid (ES)

(21) Appl. No.: 19/161,384

(22) PCT Filed: Mar. 30, 2023
(86) PCT No.: PCT/EP2023/058245

§ 371 Date: Sep. 2, 2025

(30) Foreign Application Priority Data
Mar. 2, 2023 (ES) P202330177

(51) Int. Cl. A61B 8/00

(52) U.S. Cl. CPC A61B 8/4461; A61B 8/4209; A61B 8/4281; A61B 8/461

(57) ABSTRACT

An ultrasound probe for detecting and quantifying circulating cells in superficial body fluids of an individual, comprising an ultrasound transducer; a removable membrane for engaging a target such that waves enters a superficial body fluid obtaining ultrasound data; a chamber housing a coupling material between the transducer and the membrane; and a spiral displacement mechanism changing the position of the ultrasound transducer relative to the membrane in a spiral movement around a longitudinal axis of the ultrasound probe. The spiral displacement mechanism comprises a motor; a mobile module coupled to the motor housing and slidable over a guide allowing a longitudinal displacement of the motor when actuated; and a transmission shaft transforming the rotation of the shaft of the motor into the spiral motion of the ultrasound transducer.

### FIELD OF THE INVENTION

The invention belongs to the field of ultrasound devices for medical diagnosis, triaging, screening, and treatment monitoring, and more particularly, a non-invasive ultrasound device for analysing superficial body fluids of a patient (person or animal).

### BACKGROUND OF THE INVENTION

Ultrasound waves are longitudinal compression waves of frequency greater than 20 KHz which, in the range of tenths of MHz, are useful for exploration of a sample. In general, ultrasound devices comprise a transducer in acoustic contact with the sample to be interrogated, and work by generating an ultrasonic signal through said sample and then receiving backscattered signals. From the analysis of these backscatter signals, useful information from the sample can be derived (e.g., morphology) in a non-invasive manner, hence being suitable for many applications in the medical field (e.g., imaging).

One of these applications comprises the non-invasive detection of circulating cells in superficial body fluids (mainly serous fluids), as disclosed in the patent document US2018/0263602-A1. The method disclosed therein measures circulating cells in body fluids by means of a high-frequency-based device, without the necessity of extracting a sample thereof. In this way, it serves as a diagnostic tool and for monitoring the effectiveness of a treatment administered to an individual suffering from a viral, protozoal, fungal, and/or bacterial disease. In particular, the method is useful for the diagnosis of meningeal infection and/or inflammation through the detection of circulating cells in the cerebrospinal fluid.

The method disclosed in US2018/0263602-A1 employs a high-frequency device comprising an ultrasound transducer which can be applied to the skin of the patient. Gel is usually placed between the transducer and the skin for a better acoustic impedance matching between them, reducing thus signal loss during transmission and reception. The focal point of the ultrasound to obtain an optimal signal needs to be within the target region. The ultrasound transducer of the device described in this document may comprise a single focused element, for example, a resonant flat piezoelectric ceramic or crystal and concave focusing lens. Other transducers, for example, transducers with shaped piezoelectric elements to create focusing may be alternatively used. Transducers may have different diameters (typically between 4-10 mm) and focal lengths (typically, between 5 -15 mm); being these values purely illustrative and may vary depending on the particular application.

A set of transducers, further referred to as transducer array or multi-element transducer, may also be used instead of a single transducer. Signal focalization may be obtained in this case by using geometrically focalized elements, electronic focalization (by setting a proper delay for the emission of each transducer), synthetic focalization (by postprocessing the signals obtained) or a combination of any or all of them. Once the acoustic signal or pulse is emitted by the transducer through the skin, reflected ultrasonic signals from the tissue layer interfaces as well as from cells in the fluid are received and recorded by the transducer. Next, ultrasound data are processed as a collection of, for instance, A-line data (in which the ultrasonic data are obtained in one dimension while keeping the transducer position fixed), B-mode or C-mode. B-mode and C-mode refer to 2D or 3D data representations obtained as a function of the angle and transducer position (in moving systems) respectively. 2D and 3D data can be obtained either by moving a single-element transducer, or by using fixed or moving transducer arrays (linear or 2D arrays). The 2D data representation can be shown as an image or, alternatively, to obtain quantitative measurements (e.g., flow).

The known methods of analyzing superficial body fluids require the intervention of a user (device operator) in order to vary the focal position of the probe by hand (prone to human error) or, alternatively, to use several transducers which can be turned on and off for exploring different focus lengths. However, this latter approach requires more complexity in the design of the electronics, implies a limited lateral resolution, that is the sensitivity to discriminate two points (or structures, like cells) distanced but close to each other, and only enables the scan of a discrete number of depth positions. The known methods have the following limitations:
Manual methods do not allow accurate reproducible positioning of the focus at targeted depths if cells are going to be resolved.
Electronic or synthetic focusing do enable multidepth scanning along an axial direction, but the resolution varies with depth and decreases towards the extremes of the scanned frame. Therefore, it is not possible to optimize the spatial resolution along a certain plane, which would provide a better discrimination between the cells and the noise in the measurements, thus maximizing the sensitivity to cell counting.
The use of lenses in the transducer for focusing leads to sensitivity loss because, in that case, there are different mediums with different acoustic properties.

Patent document US2012/0053468-A1 discloses a multi-focus probe. This probe is also engineered for being applied to patient skin and focus ultrasound signals within a certain region of interest (ROI). Indeed, it includes a motor coupled with a lead screw and configured to turn the lead screw about a lengthwise axis of the lead screw, wherein the lead screw includes a length having threads. The probe also comprises a lead-screw nut positioned about the lead screw such that the lead-screw nut engages the threads and such that the lead-screw nut and the lead screw can move relative to one another via the threads, a transducer configured to move vertically with the lead screw, and an enclosure surrounding the transducer. The enclosure includes a probe face configured to hold fluid and engage a wave emission target such that waves from the transducer can enter the target. Further, the probe includes a capture feature capable of engaging the lead-screw nut such that the lead-screw nut is vertically fixed relative to the probe face and such that the lead screw moves away from the probe face when rotating within the lead-screw nut in a first direction and moves toward the probe face when rotating within the lead-screw nut in a second direction opposite to the first direction while the lead-screw nut is engaged by the capture feature. The probe allows fixing a discrete number of focal depth positions for the transducer (a deep focal point operation or a shallow focal point operation), but it does not allow any arbitrary focal depth position Therefore, the vertical axial (or depth) positioning resolution is limited by the discrete levels at which the transducer may be fixed and, importantly, the transducer can only pivot about an axis perpendicular to the vertical axis to produce sector scans and in no account produce planar images at a same depth. This limits foremost the spatial resolution at a targeted depth since when performing sector (or fan-shaped) scans the distance between contiguous acoustic beams is increased with depth. The probe disclosed in patent application US 2012/0053468 A1 is therefore limited in the depth positioning resolution of the focus and in the fact that it cannot obtain planar 2D images maintaining the lateral resolution at the targeted focal distance.

All the aforementioned ultrasound devices (or probes) require the variation of the focus depth along an axial direction when it comes to detect the optimal ROI for a measurement and collect data thereof.

In this way, there is a need in this field of an ultrasonic probe able to overcome the aforementioned limitations.

### BRIEF DESCRIPTION OF THE INVENTION

As it has been outlined in the background section, a technical problem present in the field is how to accurately detect a specific region of interest (ROI) in a sample (i.e., fluid of a patient) while applying the high-frequency device (or probe) for non-invasive analysis of superficial body fluids of a target, while maintaining the maximum resolution provided by the probe across the ROI. This problem is effectively addressed by the present invention.

The invention refers to an ultrasound probe for analyzing superficial body fluids of a target. In the context of the invention, the target refers to an individual, subject or patient whose treatment is monitored with the device, the target being either a person, an animal, a recipient containing a sample or directly a sample. The operator or user of the device refers to the professional or the medical expert capable of measuring and interpreting the results derived from the use of the device. The analysis performed based on the ultrasound data acquired by the ultrasound probe can include detecting and quantifying circulating cells in a superficial body fluid of the target.

The ultrasound probe comprises:
An ultrasound transducer.
An enclosure surrounding the ultrasound transducer, wherein the enclosure includes a front part configured to engage a target such that waves from the ultrasound transducer can enter a superficial body fluid of the target via the front part to obtain ultrasound data of the superficial body fluid.
A chamber adapted to house a coupling material (e.g. a fluid) that fills the space between the ultrasound transducer and the front part.
A spiral displacement mechanism configured to change the position of the ultrasound transducer relative to the front part in a spiral movement around a longitudinal axis of the ultrasound probe.

The spiral displacement mechanism simultaneously generates an axial displacement and a rotational movement of the ultrasound transducer (i.e. a spiral displacement). The axial displacement moves the ultrasound transducer along a longitudinal axis of the ultrasound probe, towards or away from the front part of the enclosure, such that a focal volume of operation of the ultrasound transducer can be modified for analysing different depths of the superficial body fluid. The axial displacement is intended to position the focal point of the transducer within the fluid to be inspected. Moreover, the spiral displacement mechanism is configured to displace the ultrasound transducer around a rotational axis, which in combination with the axial displacement induces a spiral trajectory along the longitudinal axis of the ultrasound probe while maintaining the radiating face of the transducer orthogonal to the longitudinal axis of the ultrasound probe. Once the focal volume is located within the target fluid, the rotational movement is intended to obtain multiple signals from such fluid (or ROI).

Although a 2D plane completely perpendicular to the longitudinal axis is not possible with a spiral displacement, a sufficiently close approximation can be achieved with high resolution steps in the axial direction (which can be obtained, for instance, with threaded sections having a small thread lead). As a result, approximately the same focal depth can be considered across a complete rotation (360°) of the spiral trajectory. Advantageously, the rotational displacement enables spatial resolution optimization in each 2D plane of the ROI along the axial direction. Therefore, the spatial resolution can be adjusted to obtain multiple signals from single cells, hence, increasing the detection capabilities of the post-processed ultrasonic signal. Besides, by increasing the spatial resolution in only a portion of the 2D plane with the purpose of improving the sensitivity to single cells, the spiral displacement can still be more approximated to a radial (or circular) displacement with a negligible variation in the focal depth in that particular high-resolution plane portion. The adjustment in the spatial resolution must consider the size of the targeted cells as well as the flow of the fluid in which such cells are located. If multiple scans of the same 2D plane are needed to, for instance, compute an average of the scans, the spiral displacement mechanism can be configured to rotate the transducer in alternated clockwise and counter-clockwise directions.

The ultrasound probe is a device used for measuring and quantifying circulating cells in superficial body fluids of a target by means of high-frequency (e.g., ultrasound) transduction. The device can be used for detecting circulating cells in the fluids of an individual without the necessity of extracting a sample thereof, being useful as a screening, triaging and diagnostic tool and for monitoring the effectiveness of a treatment administered to an individual suffering from a viral, protozoal, fungal and/or bacterial disease. In certain preferred embodiments of the invention, the probe comprises an ultrasound transducer which is a single-element transducer or a multi-element (array) transducer. In a further advantageous embodiment, the array transducer consists of a Capacitive or Piezoelectric Micromachined Ultrasonic Transducer (C/PMUT). In this way, given that C/PMUTs are micromachined devices, it is easier to manufacture 2D arrays of transducers using this technology, thus favouring the integration of the rest of device electronics. Furthermore, to achieve a high frequency operation using C/PMUTs is feasible thanks to its small size.

In an embodiment, the spiral displacement mechanism comprises a motor and a transmission shaft coupled on a first end to a shaft of the motor and coupled on a second end to the ultrasound transducer. The transmission shaft is configured to transform the rotation of the shaft of the motor into spiral motion. The ultrasound transducer is preferably off-centered with regard to the shaft of the motor, so that the ultrasound transducer follows a spiral trajectory around the longitudinal axis when the motor is actuated.

In an embodiment, the transmission shaft has an outer threaded section that fits into an inner threaded section practiced on an orifice of a stopper wall attached to a housing of the ultrasound probe. The spiral displacement mechanism further comprises a mobile module coupled to a housing of the motor and slidable over a guide attached to the housing of the ultrasound probe to allow a longitudinal displacement of the motor when actuated.

The motor of the spiral displacement mechanism is preferably a stepper motor. The spiral displacement mechanism allows the ultrasound transducer to cover multiple regions of interest at a single focus depth and at higher lateral resolution than manual, electronic or synthetic focusing systems. In this way, the probe of the present invention outperforms the aforementioned techniques from the state of the art as it avoids the undesired appearance of human error in the device positioning and limited lateral resolution of alternative devices. It is worth noting that the spiral trajectory is advantageous to minimize the contact area of the probe with the targeted tissue needed for image formation, structures identification and cell counting at an acceptable measurement accuracy and precision. A minimum volume of fluid is required to obtain a representative measurement of the cell counting within such a fluid.

Advantageously, circular trajectories or spiral trajectories with high resolution steps in the axial direction are preferable because they encompass a larger volume of the ROI with a smaller scan in the plane. For instance, the ROI volume reached by a transducer trajectory sweep of 12 mm in a linear scan can be obtained with a circular trajectory in a smaller plane path (4 mm). This is a crucial advantage when it comes to analyze cerebrospinal fluid white blood cells through an infant's fontanel, as fontanelles close progressively after birth (typically taking between 12-18 months), so the available surface for the planar scan decreases in time.

In further embodiments where the coupling material is a fluid, the front part of the enclosure comprises a removable and/or replaceable membrane configured to make contact with the target. The fluid is arranged inside the chamber, between the ultrasound transducer and the membrane. The membrane is a consumable element. In this way, the membrane is fungible, and it is replaced for use in each patient, to maximize acoustic coupling, improve the sensitivity, avoid the fluid from spilling out of the chamber, reduce cross-contamination and ensure proper performance. In further preferred embodiments, the membrane is made of polyvinyl alcohol (PVA). In this way, hydrogels like PVA enable proper acoustic coupling with the patient body without the need of gel. Otherwise, the use of plastic-base materials like plastic films for the membrane requires ultrasound gel for proper coupling, and provides a worse acoustic coupling, in addition to amplifying unwanted artifacts.

The spiral displacement mechanism provides, through a single stepper motor implemented in the probe, an automatic combined longitudinal and rotational displacement of the ultrasound transducer, offering not only a more precise control of the longitudinal and rotational movements, but also a highly compact, light and robust mechanism translated in a better usability and measurement quality. This configuration also contributes to an easier assembly protocol of the probe, allowing to change critical parts independently of the rest of the mechanism.

In some embodiments of the invention, the probe is placed on a probe stabilization support that is in contact with the patient which helps maintaining the probe in a stable position to guarantee an adequate coupling of the probe onto the patient during the measurement. Ideally, this support perfectly adapts to the shape of the tissue of the patient, due to the flexibility of the material it is made of. Additionally, the geometry of the support prevents the user from exerting an excessive pressure on the patient during the measurement. Therefore, in such type of embodiments the device also performs a monitoring function in which frequent measurements can be obtained to track the evolution of the measured parameter, e.g. cells, as a response of the patient to the administered treatment.

The object of the invention can be used as a stand-alone device, or alternatively, it can be integrated in a system along with a conventional ultrasound equipment (which in turn comprises electronics for beamforming, and a sensor array). In both cases, additional electronics is required for controlling, synchronizing, and triggering the stepper motor of the device. In the stand-alone case, most of these electronics can be housed within a support base attached to the device. On the other hand, when the device is integrated in a commercial ultrasound equipment, then the base can be embedded in said commercial equipment. Furthermore, in this latter case the stepper motor of the device is also synchronized and triggered according to the signals acquired by the commercial ultrasound equipment.

In this way, the electronics housed in the base provides high spatial and temporal resolution to allow applications involving cell counting, and comprises a high frequency ultrasound (e.g., 20 MHz) enabling high amplitude emission, a transducer signal preamplifier (20-25 dB) providing a high sensing gain, hardware and software signal filters (including filters for performing operations such as averaging, removing Electro-Magnetic Interference, pass band selection, correlation, etc.) in order to enhance signal-to-noise (SNR) ratio. Furthermore, additional adjustable gain filters can be included to provide tuneable gain depending on the tissue to be explored (i.e., according to its attenuation). For instance, the gain obtained with the transducer preamplifier can be complemented by a digital (software) gain applied to the signal after its digitalization a post-processing step. By considering a digital gain of 55 dB, the resulting gain that can be obtained is 80 dB.

In certain embodiments, the device further comprises a display installed at the support base or at the probe. This display can be used to show meaningful information obtained from the backscattered ultrasound signals, for instance, information for guiding the operator (e.g. correct positioning, coupling).

In particular applications of the present invention, the device is used for superficial body fluids screening, for instance, to detect certain infections and diseases through ultrasound analysis of said fluids. In a more preferred application, it can be used for counting cerebrospinal fluid (CSF) white blood cells through the infant's fontanel, as an indicator that allows diagnosing bacterial meningitis, among other diseases. This life-threatening infection causes several thousands of deaths per year worldwide, and often leaves lifelong effects since its prognosis worsens with delayed treatments. However, this is in general a challenging task, due to the minimal symptomatology of the disease especially in newborns and young infants who are more vulnerable and limited in their ability to communicate, and sometimes a fever is the only symptom visible to the naked eye. In this context, early detection of this disease can be lifesaving, and the invention can contribute thereof. The device can be placed, for instance, over the fontanel of the infant where bones remain unclosed, where it can effectively measure the number of white blood cells in the CSF, avoiding the need for a lumbar puncture. Thus, the device allows for easier and earlier diagnosis of meningitis which translates into a decrease in the mortality rates due to this disease. In the scope of this application, it is worth underlining the different role played by the longitudinal and rotational movement of the device. The longitudinal movement serves for fontanel tissue characterization (through thickness and attenuation) and searching of the best position for measuring the CSF. On the other hand, the rotational movement is required for image formation, for identifying different structures of the patient body and for cell counting (a sufficient volume needs to be inspected to have significant statistics). The spatial resolution used for cell counting is adjusted according to the size of the targeted cells as well as the flow of the fluid in which such cells are located.

The device and/or the system described above can be also used for imaging applications as a high-resolution ultrasound, so the data obtained by the device and/or the system are shown to the user as an image. The device may also be used to measure other parameters, such as tissue thickness.

In the scope of this invention, the term “superficial body fluid” refers to serous fluids comprising, among others: cerebrospinal fluid, synovial fluid, peritoneal fluid, pericardial fluid, pleural fluid, amniotic fluid and aqueous humor. The invention can be used for all the aforementioned cases. The blood is explicitly excluded from this term. These superficial body fluids are substantially transparent from the acoustic point of view for the ultrasonic waves, such that the concentration changes of the cells flowing within them are significant.

### BRIEF DESCRIPTION OF THE DRAWINGS

To complete the description and in order to provide for a better understanding of the invention, a set of drawings is provided. Said drawings form an integral part of the description and illustrate an embodiment of the invention, which should not be interpreted as restricting the scope of the invention, but just as an example of how the invention can be carried out. The drawings comprise the following figures:

FIG. 1 shows a detailed section of the internal structure of the ultrasound probe.

FIGS. 2A-2C illustrate the planar displacement of the ultrasound transducer from a front view and in three different positions, when a clockwise rotation is performed.

FIGS. 3A-3C show a lateral section view of the ultrasound probe, to further illustrate how the motor of the spiral displacement mechanism transmits the spiral movement to the mobile module, transmission shaft and ultrasound transducer. Three different positions (front, middle, rear) of the spiral displacement mechanism are displayed.

FIGS. 4A-4C show a zoomed-in view of FIGS. 3A-3C, and more particularly, a view of the second body comprising the ultrasound transducer, the removable cap and the membrane.

FIGS. 5A-5D illustrate a process for filling the fluid chamber of the ultrasound probe with water.

FIGS. 6A-6E show the longitudinal and rotational displacement of the ultrasound transducer for fontanel tissue characterization.

FIGS. 7A-7C depict an embodiment of the probe stabilization support used to keep the ultrasound probe in a steady position.

FIGS. 8A-8C depict an embodiment of a system for analyzing superficial body fluids, the system comprising the ultrasound probe and a support base.

### DETAILED DESCRIPTION OF THE INVENTION

Here, we describe possible implementations of the device, which is the main element of the object of the invention. It is important to understand that following embodiments only intend to illustrate particular designs of such device.

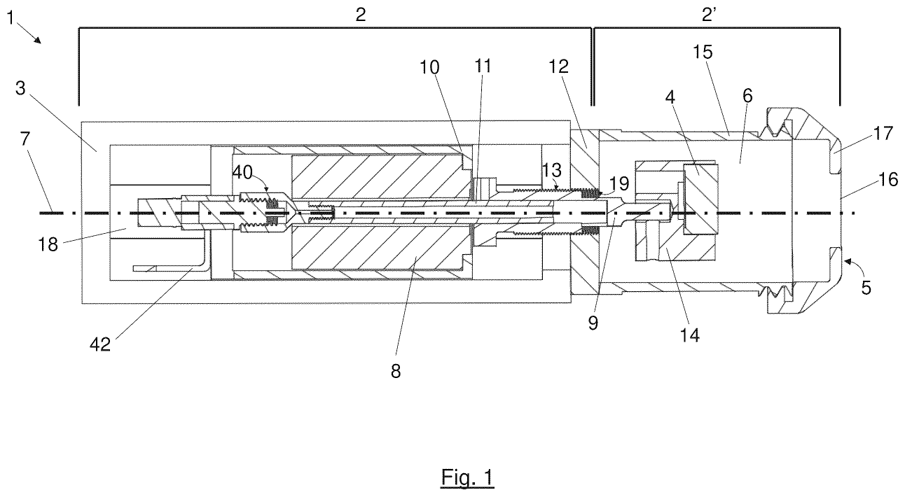

FIG. 1 represents a cross section view of an ultrasound probe (1) for analyzing superficial body fluids of a target, according to an embodiment.

The ultrasound probe (1) depicted in FIG. 1 comprises a first body (2) and a second body (2′) connected to each other (e.g., threaded) and housing the components of the probe. Alternatively, the ultrasound probe (1) may be implemented with a different number of bodies (e.g., one body or more than two bodies). The first body (2) shown in FIG. 1 comprises a housing (3) and a spiral displacement mechanism and the second body (2′) houses an ultrasound transducer (4) configured to focus on a superficial body fluid of a target to obtain ultrasound data of the superficial body fluid. The ultrasound transducer (4) is surrounded by an enclosure. A front part (5) of the enclosure is adapted to make contact with an external part of a target (e.g., skin of person or animal) such that waves from the ultrasound transducer (4) can enter a superficial body fluid of the target via the front part (5) to obtain ultrasound data of the superficial body fluid. The enclosure defines a chamber (6) prepared to house a coupling material (e.g., a fluid material such as water) that fills the space between the ultrasound transducer (4) and the front part (5) of the ultrasound probe (1).

The spiral displacement mechanism is coupled to the ultrasound transducer (4) and is configured to change the position of the ultrasound transducer (4) relative to the front part (5) of the enclosure in a spiral movement around a longitudinal axis (7) of the ultrasound probe (1). The spiral displacement mechanism is configured to displace the position of the ultrasound transducer (4) along the longitudinal axis (7) of the ultrasound probe (1), so that the depth of the focal point (or region) of analysis of the ultrasound transducer (4) can be modified for analysing different depths of the superficial body fluid. The spiral displacement mechanism moves the ultrasound transducer (4) towards or away from the front part (5). Simultaneously with the longitudinal displacement, the spiral displacement mechanism is also configured to rotate the ultrasound transducer (4) around the longitudinal axis (7), such that the ultrasound transducer (4) follows a spiral trajectory around the longitudinal axis (7).

Advantageously, the spiral displacement mechanism moves the ultrasound transducer (4) in the rotational and longitudinal direction. In this way, the ultrasound probe (1) combines planar and axial sweep for scanning a region of interest. First, the axial sweep enables a proper positioning in a certain plane for performing the measurement. Then, the planar scanning provides the region where there is a minimum volume of cells for acquiring a precise measurement.

In an embodiment, the spiral displacement mechanism comprises a stepper motor (8) coupled to a transmission shaft (9), which is responsible for generating the axial and rotational displacement (i.e. spiral displacement) of the ultrasound transducer (4).

The housing (3) is the main structure of the device inside of which most of the elements of the ultrasound probe (1) are arranged. It is preferably manufactured with aluminium to give consistency and lightness to the structure. The spiral displacement mechanism is arranged in the housing (3) and comprises a mobile module (10) coupled to the housing of the stepper motor (8) and that slides over a guide (18) practiced on (or attached to) the housing (3) of the ultrasound probe (1) to enable a longitudinal displacement of the stepper motor (8) and a spiral displacement of the transmission shaft (9) and the ultrasound transducer (4).

In the embodiment depicted in FIG. 1 the spiral displacement mechanism comprises the transmission shaft (9) coupled to the shaft (11) of the stepper motor (8) via a captive screw. The transmission shaft (9) is involved in the transformation of the rotational motion of the stepper motor (8) into a spiral motion, having an outer threaded section (13) that fits into the inner threads of an inner threaded section (19) practiced on an orifice of a stopper wall (12) attached to a housing of the first body (2) or the second body (2′). In this way, the mobile module (10) is able to move along the axial direction, parallel to the longitudinal axis (7), forced by the spiral movement of the transmission shaft (9) when the stepper motor (8) is actuated. The transmission shaft (9) is preferably manufactured with aluminium.

The mobile module (10) is the other piece that participates in the conversion of rotational motion into linear motion. The stopper wall (12) has a threaded section (19) that fits in with the threaded section (13) of the transmission shaft (9), so that the sliding between the threads of both elements forces the linear displacement of the mobile module (10) towards the front or the rear of the ultrasound probe (1), depending on the direction of rotation of the stepper motor (8). The front part of the mobile module (10) serves as a union with the stepper motor (8). This mobile module (10) is preferably made of PEEK, which is a resistant and biocompatible plastic material exhibiting a low coefficient of friction in order to guarantee a smooth glide over the metal components that surround it, in particular the housing (3).

In an embodiment, the ultrasound probe (1) comprises an electronic axial displacement blocking module that includes a plate (42) coupled to the mobile module (10) and an optoelectronic system, based on one or more photodetectors (not shown in FIG. 1), which is attached to the housing (3). When moving the mobile module (10), the plate (42) crosses the photodetector at an innermost axial displacement of the axial trajectory, which in turn detects the presence of the plate (42) and produces an electronic signal used to stop the stepper motor (8) in order to limit the motion of the mobile module (10). The axial displacement blocking system is intended to prevent the ultrasound transducer (4) from getting too close to the chamber (6) boundaries (e.g., to detect an innermost axial displacement to avoid getting too close to the front part (5) of the enclosure) and being damaged. The axial displacement blocking system may also be used to prevent the ultrasound transducer (4) from getting too far from the front part (5) of the enclosure.

The enclosure surrounds the chamber (6) and is coupled (preferably screwed) to the front of the housing (3). A transducer holder (14), which houses or holds the ultrasound transducer (4), is fixed (e.g., with a captive screw) to the transmission shaft (9).

The front part (5) of the enclosure constitutes the tip of the ultrasound probe (1) configured to contact the target. Although the enclosure may be formed by one piece made of the same material, in the preferred embodiment shown in the figures the enclosure is formed by several pieces joined together, made of different materials. In particular, the enclosure may comprise a chamber body (15) and a front part (5). The front part (5) preferably includes a membrane (16) configured to make contact with the target. A removable cap (17) is configured to fix the membrane (16) to the chamber body (15). The chamber body (15) is preferably manufactured with PEEK to provide consistency to the chamber (6) while remaining light.

The transducer holder (14) is attached to the transmission shaft (9) and holds the ultrasound transducer (4). The ultrasound transducer (4) is slightly off-axis about the shaft (11) of the stepper motor (8) (or, in FIG. 1, the longitudinal axis (7) of the ultrasound probe (1)) so that when the stepper motor (8) is activated the ultrasound transducer (4) describes a spiral trajectory around the longitudinal axis (7). When using a small thread pitch (e.g. not greater than 0.5 mm) in the threaded section (13) of the transmission shaft (9), a complete turn (360°) of the ultrasound transducer (4) is almost contained on a plane perpendicular to the longitudinal axis (7). The transducer holder (14) is preferably made of POM (polyoxymethylene) or a 3D print material, which is an easily machinable and durable plastic material.

The function of the ultrasound transducer (4) is emitting and receiving the ultrasound signal. Particularly, it is a focused single-element transducer with high spatial resolution, thus allowing for detection of small particles flowing in liquids.

The membrane (16) fits with the chamber body (15) boundaries. In an embodiment, the membrane (16) is manufactured (at least partially) with hydrogel material, preferably polyvinyl alcohol (PVA). By using this hydrogel, the membrane (16) exhibits great flexibility and resistance, whence it enhances the acoustic coupling between the device and the patient body during the measurement. On the other hand, this hydrogel exhibits a low attenuation level, and consequently, excellent ultrasonic signal transmission capabilities. Alternatively, the membrane (16) may be made (at least partially) of other material, such as plastic film. The membrane (16) is removable and can be replaced after each use or measurement; the membrane (16) is therefore a consumable element.

The removable cap (17) is preferably made of PEEK, which is a biocompatible material in addition to aforementioned properties. The removable cap (17) fixes the membrane (16) to the chamber body (15). The geometry of the removable cap (17) is designed so that only the membrane (16) is in contact with the patient during the measurement. In this way, the removable cap (17) does not affect the coupling of the device.

As a summary, the device displayed in FIG. 1 has a spiral displacement mechanism, with rotational and axial displacement actuated by one stepper motor (8). The axial displacement allows the positioning of the ultrasound transducer (4) so that the focus is placed in a region of interest (ROI) of the patient body. On the other hand, the rotation enables the transducer to scan the ROI volume required for the measurement.

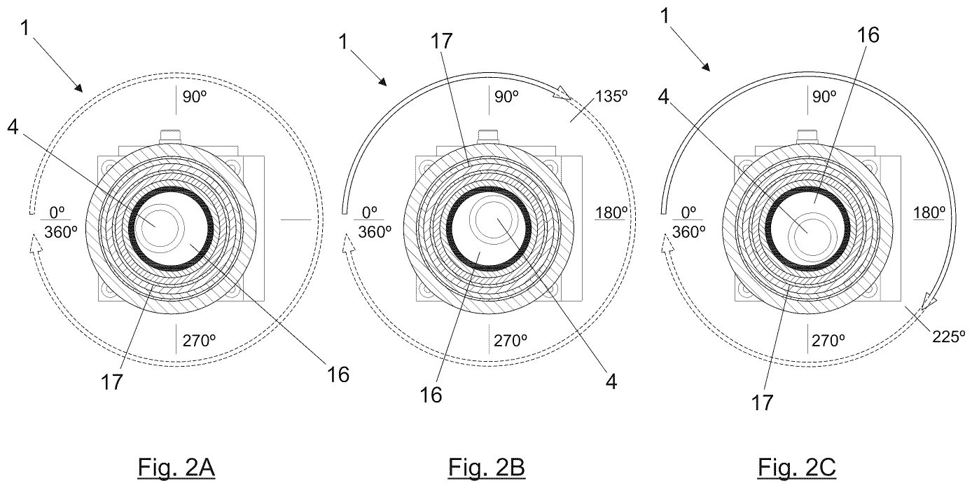

FIGS. 2A-2C show three different instants during the spiral displacement of the ultrasound transducer (4) from a front view of the ultrasound probe (1) of FIG. 1. In this front view the ultrasound transducer (4), which is off-centered from the longitudinal axis (7) of the device, describes a circular trajectory around the longitudinal axis (7) (e.g., of 2 mm radius).

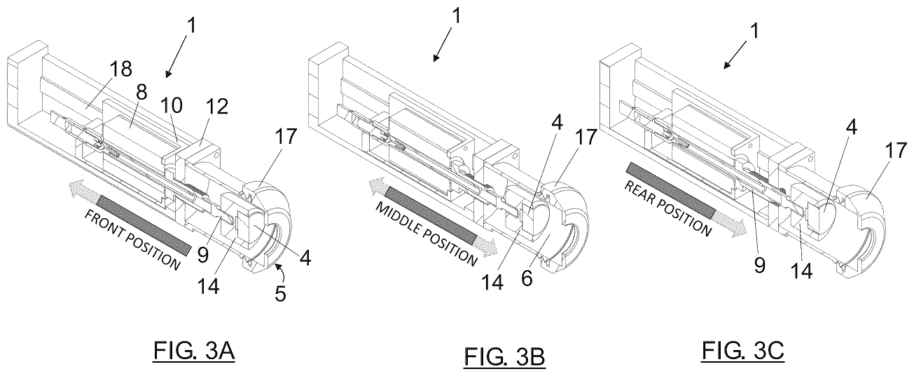

FIGS. 3A-3C display, in a lateral section view of the ultrasound probe (1), three different positions in the axial displacement of the stepper motor (8) and the spiral displacement of the ultrasound transducer (4). The stepper motor (5) allows the mobile module (10) to slide over the housing (3) along the longitudinal axis (7) of the device. The transmission shaft (9) transmits the spiral displacement to the ultrasound transducer (4), allowing the ultrasound transducer (4) to get closer or further to the target (or to the membrane (16) arranged at the tip of the device, when using a fluid coupling material). In particular, these figures depict a front position (FIG. 3A), a middle position (FIG. 3B) and a rear position (FIG. 3C) of the ultrasound transducer (4) with regard to the front part (5).

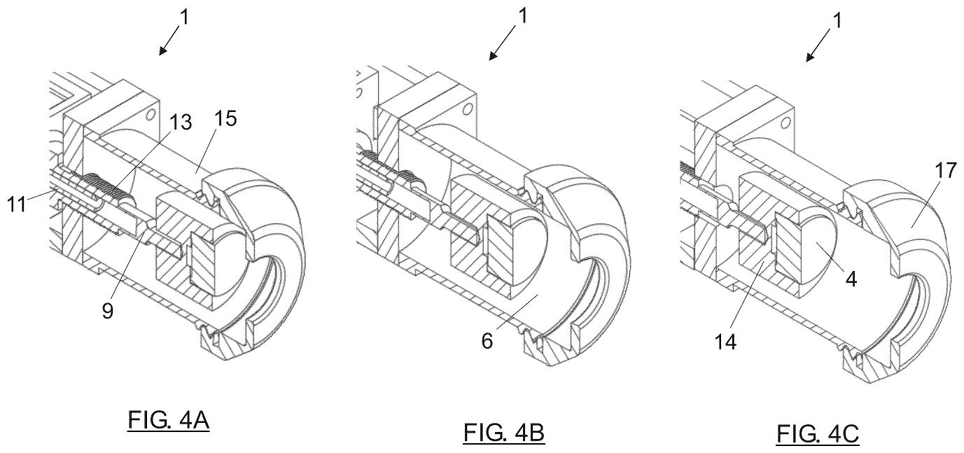

FIGS. 4A-4C is a zoomed-in view of FIGS. 3A-3C to show in more detail the axial displacement of the ultrasound transducer (4). In the embodiment depicted in FIGS. 4A-4C, the axial displacement allows the ultrasound transducer (4) to describe a 10 mm trajectory along the longitudinal axis (7); in particular, the ultrasound transducer (4) can be positioned between 2 mm and 12 mm from the membrane (16), which is not shown in these figures. The membrane (16) is preferably made of a hydrogel that guarantees an optimal coupling between the device and the patient body in order to eliminate the need of coupling gel between the membrane (16) and the patient body. The removable cap (17) is preferably made of PEEK and is used to fix the membrane (16) and avoid leakage from the chamber (6) to the patient body.

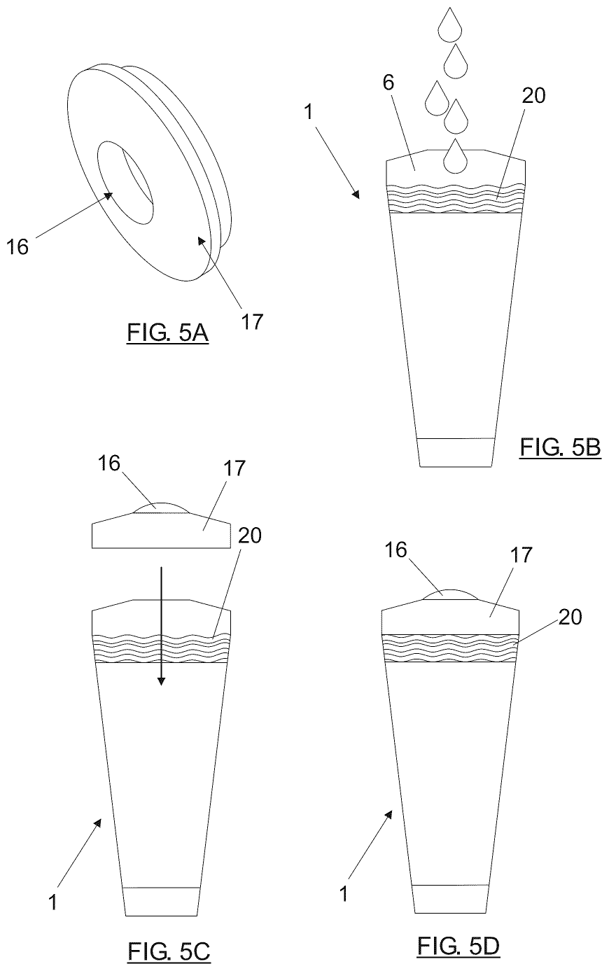

FIGS. 5A-5D depicts a process for filling the chamber (6) with distilled water, according to an embodiment. In these figures the ultrasound probe (1) is represented schematically, its shape not corresponding with the shape of the ultrasound probe (1) shown in the previous figures. First, the removable cap (17) is removed from the ultrasound probe (1), along with the membrane (16). The removable cap (17), already removed from the ultrasound probe (1), is shown in FIG. 5A. Afterwards, the chamber (6) of the ultrasound probe (1) is filled with water (20), preferably distilled water (FIG. 5B). Then, the removable cap (17) and the membrane (16) are placed back on the probe (FIG. 5C). The removable cap (17) may be fixed to the ultrasound probe (1) in multiple ways (e.g. threaded, press fitted, etc.). FIG. 5D depicts the ultrasound probe (1) with the removable cap (17) already fitted.

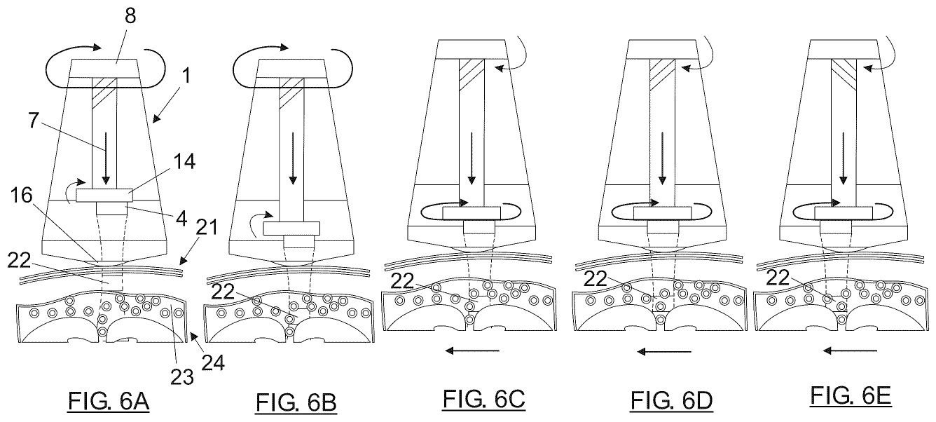

FIGS. 6A-6E depicts the spiral displacement of the ultrasound transducer (4) when applied to a baby's head for fontanel tissue characterization. As in the previous figure, the ultrasound probe (1) is depicted schematically.

Firstly, the ultrasound probe (1) is applied on a surface of a target, in this case a baby's head (21). Initially, as shown in FIG. 6A, the focal volume (22) in which the backscattered signals amplitude is maximum is outside the superficial body fluid (23), in this example the target is cerebrospinal fluid of fontanel tissue (24). In an embodiment, the spiral displacement mechanism of the ultrasound probe (1) is actuated automatically (e.g. by analysing the ultrasound data and using image recognition), to axially displace the ultrasound transducer (4) towards the membrane (16) along the longitudinal axis (7).

FIG. 6B shows the position of the ultrasound transducer (4) after the axial displacement, when the focal volume (22) is already within the target volume that includes the superficial body fluid (23). However, for a better characterization of the fontanel tissue (24) a search on a greater focal volume (22) is required for cell counting, since a sufficient volume needs to be inspected to have significant statistics when trying to detect and quantify circulating cells in superficial body fluids and identify structures with an acceptable precision.

FIGS. 6C-6E shows the focal volume (22) being moved almost on a plane perpendicular to the longitudinal axis (7), when actuating the stepper motor (8) one turn (360°) and using a transmission shaft (9) with a small thread pitch in the threaded section (13). The ultrasound transducer (4) describes a circular trajectory almost contained on a plane perpendicular to the longitudinal axis (7); likewise, the focal volume (22) describes a similar trajectory inside the superficial body fluid (23), and a larger volume is thus scanned.

The main advantages of the embodiment shown in previous figures comprise:
The axial displacement through an automatic mechanism, compared to other methods such as a gear mechanism or a lever mechanism with fixed positions:
   a. It occupies a reduced space, which allows that the device does not require an excessive length, thus favouring the device ergonomics and quality of the data.
   b. It provides high precision and enables axial displacements of only a few tenths of a millimetre, thus being able to choose the ROI with high accuracy.
   c. It exhibits a high pushing force to overcome the friction that occurs between the different elements of the device due to rotation and axial displacement.
The transmission of almost planar movement around a longitudinal axis of the device, which outperforms other techniques such as a swing platform mechanism because:
   a. It allows optimising the lateral resolution to maximise the amplitude and number of signals reflected by small (micron-sized) structures, e.g. cells, located in the focal region.
   b. It occupies a smaller area which favours a thinner device design, thus improving the device ergonomics.
   c. It generates a minor volume change in the chamber (6) when the spiral displacement is activated, hence preventing the membrane from excessive bulging or retraction.

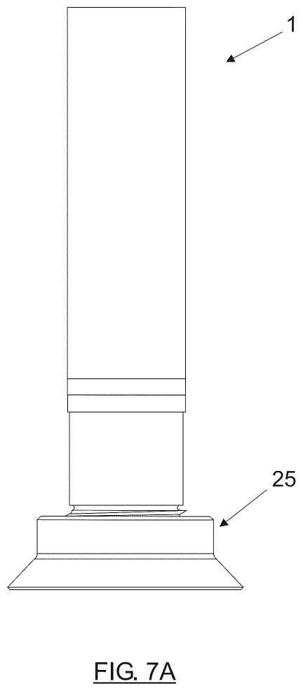

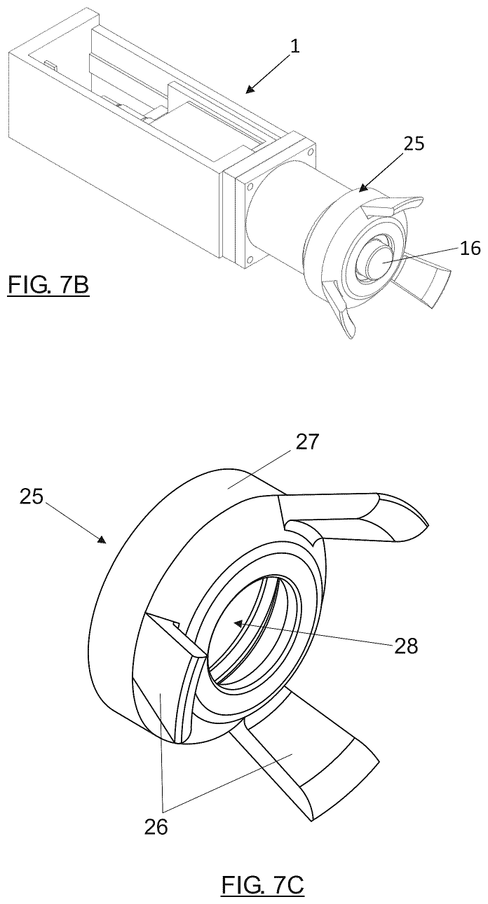

FIGS. 7A and 7B show the ultrasound probe (1) attached to a probe stabilization support (25) that allows the user to keep the probe in a still position while preventing the excessive pressure put on the tissue of the patient. The probe stabilization support (25) shown in these figures is a tripod-shaped tip adapted to accommodate the weight of the probe on the patient's head in order to provide stability at the time of measurement. The probe stabilization support (25) may include a plurality of flexible wings (26) which allows to adapt to the shape of the measurement spot (e.g. the head) on the patient. In an embodiment, the probe stabilization support (25) is made of a flexible 3D-printed composite material (Vero White Plus and Agilus Black) using Polyjet technology. The probe stabilization support (25) is aimed to rest on the patient body, close to the location of the optimal measurement spot on the patient tissue, and allows the user to hold the probe still and keep it in a stable position while the measurement is being performed. Additionally, it prevents the user from exerting excessive pressure over the patient while relieving the force that the user needs to do to hold the probe, thus improving the coupling of the ultrasound probe (1) to the patient. It is made of a flexible material that can adapt perfectly to the shape of the patient, improving the stability and usability of the probe.

FIG. 7C depicts the probe stabilization support (25) detached from the ultrasound probe (1), which includes a removable frame (27) adapted to be attached or detached from the enclosure of the ultrasound probe (1). The frame (27) has a central orifice (28) thorough which the membrane (16) protrudes to contact the body of the target. The shape of the probe stabilization support (25) is adapted to the contact area so that it can rest on the target and provide stabilisation during the acquisition of the ultrasound data. To that end, the probe stabilization support (25) may include one or more flexible wings (26).

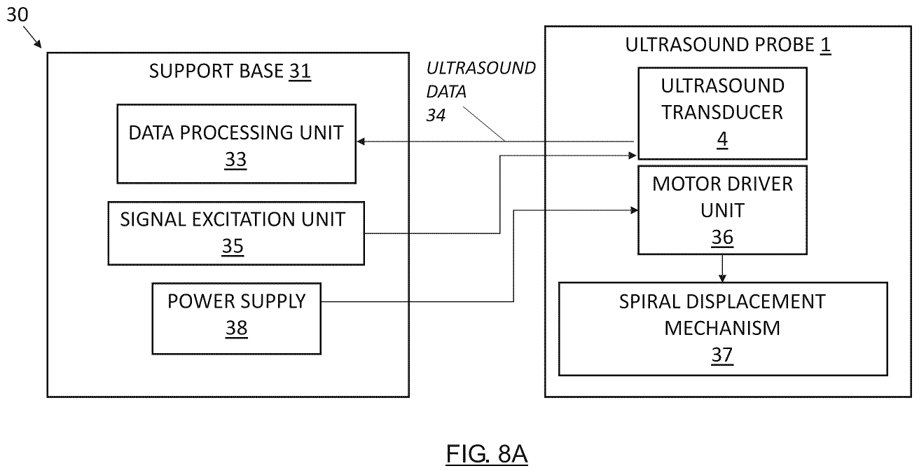

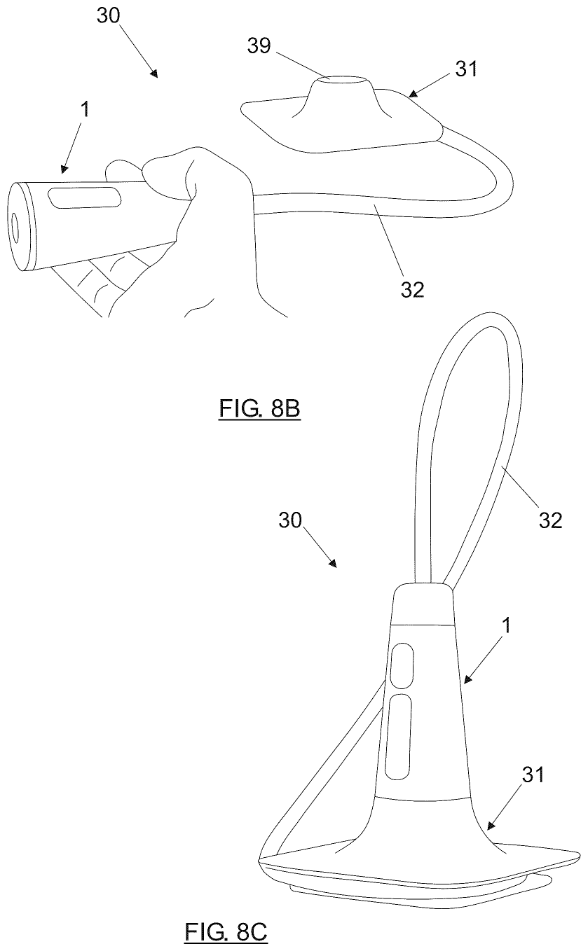

The present invention also refers to a system (30) for analyzing superficial body fluids of a target. FIGS. 8A-8C depict an embodiment of the system (30). According to this embodiment, the ultrasound probe (1) is coupled to a support base (31) that serves as a support and further comprise additional electronics for actuating the ultrasound probe (1). The support base (31) is connected to, and in communication with, the ultrasound probe (1) by wire (32). The support base (31) houses several electronic components depicted in the schematic layout of FIG. 8A; in particular:
A data processing unit (33) configured to acquire ultrasound data (34) obtained by the ultrasound probe (1) applied on a target. The ultrasound data (34) may then be stored on a memory. The data processing unit (33) may be configured to analyse the acquired ultrasound data (34) and correlate the analysed information with the presence and amount of circulating cells in the target. Alternatively, or in addition, the analysis and correlation may be performed remotely or the results may be sent to a local or remote server. To that end, the support base (31) may include a communication module (e.g. Wi-Fi module) configured to send the ultrasound data (34) and the measurement to a remote server for further analysis.
A signal excitation unit (35) configured to control the actuation of the ultrasound transducer (4).
A motor driver unit (36) configured to control the spiral displacement mechanism (37) of the ultrasound probe (1).
A power supply (38) configured to provide energy to the electronic components included in the support base (31) and the ultrasound probe (1).

The ultrasound probe (1) is engageable to an orifice (39) practiced on the support base (31), shown in FIG. 8B. The orifice (39) has a size such that the front of the ultrasound probe (1) fits into the orifice (39), as depicted in FIG. 8C. The support base (31) may further comprise a display (not shown in the figures) to show information regarding the presence and concentration of circulating cells in the target.

The support base (31) may further include a control unit in charge of controlling the different components of the support base (31). Alternatively, the control unit may be integrated in the data processing unit (33). Regarding the actuation of the spiral displacement mechanism (37), the motor driver unit (36) may be configured to automatically control the spiral displacement mechanism (37) to generate a spiral movement of the ultrasound transducer (4) (alternatively rotating clockwise and counter-clockwise). The spiral displacement of the ultrasound transducer (4) may also be performed automatically by integrating image recognition (from ultrasound data acquired by the ultrasound transducer (4)) with machine learning models. Alternatively, the spiral displacement may be performed by the user through a user interface (e.g. input means such as switches, graphical user interface, etc.).

The embodiment depicted in FIG. 1 is composed of a transducer holder (14) that embraces the transducer (4) from its sides and back, leaving the active face of the transducer on the front uncovered. The back side of the transducer holder (14) contains an orifice out of which the transducer signal cable (not shown in FIG. 1) leaves to pass through the transmission shaft (9) that connects the transducer holder (14) and the motor (8). The motor (8) has an orifice that goes all along its central axis. When leaving the transmission shaft (9), the transducer signal cable passes through the central axis of the motor (8). The transducer signal cable therefore passes inside the transmission shaft (9) and the motor shaft (11).

The end of the transducer signal cable that leaves the end of the motor (i.e. the motor side that is furthest from the transducer (4)) is connected to a coaxial connector system in charge of transmitting the signal from the transducer (4) and, at the same time, isolating the spiral motion of the transducer (4). The connector system includes a rotatory element (40) to where the transducer signal cable is connected. The rotatory element (40) is screwed in the motor shaft (11) and connects to a second component of the connector system. The second component is a fix connector (not shown in FIG. 1) electrically connected with the rotatory element by means of a connector pin, but mechanically uncoupled from the rotatory movement.

In another embodiment, the electronic components previously described for the support base (31) may be directly integrated in the ultrasound probe (1) or in a base directly attached to the ultrasound probe (1), thus eliminating the external wire (32).

The key to achieve an early diagnosis of diseases though the analysis of superficial body fluids (as, for example, meningitis in the CSF) is the detection of individual cells, in order to get an in vivo efficient measurement of cell concentration at low concentration ranges (1-1,000 cells/μL). In this context, the ultrasound probe of the present invention is used to acquire ultrasound data (34) for the analysis of superficial body fluids using any method, such as the method for detecting circulating cells in superficial body fluids disclosed in patent document US 2018/0263602 A1.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An ultrasound probe for analyzing superficial body fluids, the ultrasound probe comprising:
an ultrasound transducer;
an enclosure surrounding the ultrasound transducer, wherein the enclosure includes a front part configured to engage a target such that waves from the ultrasound transducer can enter a superficial body fluid of the target via the front part to obtain ultrasound data of the superficial body fluid;
a chamber adapted to house a coupling material that fills space between the ultrasound transducer and the front part; and
a spiral displacement mechanism configured to change the position of the ultrasound transducer relative to the front part in a spiral movement around a longitudinal axis of the ultrasound probe.

2. The ultrasound probe according to claim 1, wherein the spiral displacement mechanism comprises:
a motor, and
a transmission shaft coupled on a first end to a shaft of the motor and coupled on a second end to the ultrasound transducer, wherein the transmission shaft is configured to transform the rotation of the shaft of the motor into spiral motion.

3. The ultrasound probe according to claim 2, wherein the ultrasound transducer is off-centered with regard to the shaft of the motor so that the ultrasound transducer follows a spiral trajectory around the longitudinal axis when the motor is actuated.

4. The ultrasound probe according to claim 2, wherein the transmission shaft has an outer threaded section that fits into an inner threaded section practiced on an orifice of a stopper wall attached to a housing of the ultrasound probe;
and wherein the spiral displacement mechanism further comprises a mobile module coupled to a housing of the motor and slidable over a guide attached to the housing of the ultrasound probe to allow a longitudinal displacement of the motor when actuated.

5. The ultrasound probe according to claim 1, wherein the spiral displacement mechanism comprises an electronic axial displacement blocking module including at least one photodetector configured to:
detect at least one innermost or outermost axial displacement of any of the mobile module, the transmission shaft or the ultrasound transducer; and
generate an electronic signal to stop the motor when an innermost or outermost axial displacement is detected.

6. The ultrasound probe according to claim 1, wherein the front part of the enclosure comprises a removable membrane configured to contact the target.

7. The ultrasound probe according to claim 6, wherein the membrane is made of polyvinyl alcohol.

8. The ultrasound probe according to claim 6, wherein the membrane is made of plastic film.

9. The ultrasound probe according to claim 6, further comprising a removable cap configured to fix the membrane to the chamber

10. The ultrasound probe according to claim 1, wherein the ultrasound transducer is a single-element transducer, a multi-element transducer, a CMUT or a PMUT transducer.

11. The ultrasound probe according to claim 1, further comprising a probe stabilization support including:
a removable frame adapted to be attached to the enclosure of the ultrasound probe the frame having a central orifice such that the front part of the enclosure can engage a contact area of a target;
a plurality of flexible wings configured to engage the target around the contact area so that the probe stabilization support can rest on the target to provide stabilisation during the acquisition of ultrasound data.

12. The ultrasound probe according to claim 1, for use in analysis of a serous fluid selected from the group consisting of: cerebrospinal, synovial, peritoneal, pericardial, pleural, amniotic and aqueous humor.

13. The ultrasound probe according to claim 1, for use in image applications.

14. A system for analyzing superficial body fluids, the system comprising an ultrasound probe according to claim 1 and a support base connected to the ultrasound probe, wherein the support base includes:
a data processing unit configured to acquire ultrasound data obtained by the ultrasound probe applied on a target, analyse the acquired ultrasound data and correlate the analysed information with the presence and amount of circulating cells in the target;
a signal excitation unit configured to control the actuation of the ultrasound transducer;
a motor driver unit configured to control the spiral displacement mechanism of the ultrasound probe; and
a power supply.

15. The system according to claim 14, wherein the ultrasound probe is engageable to an orifice of the support base.

16. The system according to claim 14, wherein the support base further comprises a display to show information regarding the presence and amount of circulating cells in the target.
